(19) United States
(12) Patent Application Publication
(10) Pub. No.: US 2010/0234315 A1
(43) Pub. Date: Sep. 16, 2010

(54) COMPOSITIONS AND METHODS FOR TREATING PSYCHIATRIC AND BEHAVIORAL DISORDERS

(76) Inventor: PAUL HERSCU, AMHERST, MA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/721,678

(22) Filed: Mar. 11, 2010

Related U.S. Application Data
(60) Provisional application No. 61/159,568, filed on Mar. 12, 2009.

(51) Int. Cl. A61K 31/7028; A61K 31/4375; A61K 31/445; A61P 25/22; A61P 25/30; A61P 25/20; A61P 25/24; A61P 25/00

(52) U.S. Cl. 514/34; 514/279; 514/319

(57) ABSTRACT

In one aspect, the present invention provides methods that comprise administering to a patient an active agent that comprises one or more of protoveratrine A, protoveratrine B, cevadine, veratramine and veratrosine in an amount that is effective to lessen the severity or occurrence of at least one symptom indicative of the disorder.

### CROSS-REFERENCE TO RELATED APPLICATION

This application claims the benefit of U.S. Application No. 61/159,568 filed Mar. 12, 2009, which is incorporated by reference herein in its entirety.

### TECHNICAL FIELD

The present invention relates generally to the treatment of psychiatric and behavioral disorders such as attention deficit/hyperactivity disorder (ADHD) and attention deficit disorder (ADD).

### BACKGROUND

Treatments for psychiatric and behavioral disorders vary from cognitive and behavioral therapy such as speech therapy, physical therapy or occupational therapy, to the use of various drugs such as methylpheidate and amphetamines. Many of the drugs used have undesirable side effects such as the development of tic disorders, the increase in blood pressure or heart arrhythmia, or the loss of appetite. There thus remains a need for an effective drug to treat psychiatric and behavioral disorders such as ADHD and ADD that will not lead to such side effects.

### SUMMARY

In one aspect, the present invention provides methods for treating patients that suffer from a psychiatric disorder or a behavioral disorder such as, for example, ADD, ADHD or anxiety disorder. Preferred methods of this type involve administering to such a patient an active agent that comprises one or more of protoveratrine A, protoveratrine B, cevadine, veratramine and veratrosine in an amount that is effective to lessen the severity or occurrence of at least one symptom indicative of the disorder. The patient can optionally be examined before and/or after administering the active agent to determine the severity or occurrence of at least one symptom indicative of the disorder to, for example, assess the effectiveness of the agent.

The present invention also provides pharmaceutical dosage forms comprising protoveratrine A in an amount that is no more than about 0.9 mg/kg. The present invention also provides pharmaceutical dosage forms comprising protoveratrine B in an amount that is no more than about 1.0 mg/kg.

### BRIEF DESCRIPTION OF THE DRAWINGS

FIG. 1 illustrates mean beam breaks by strain: baseline (filled), 0.040 mg/kg (open), 0.020 mg/kg (light gray), 0.010 mg/kg (dark gray). The time in minutes is shown on the y-axis.

FIG. 2 illustrates the mean beam breaks by strain corresponding to the first 30 bins of sessions. The dose of protoveratrine B in mg/kg is shown on the y-axis.

FIG. 3 illustrates the mean rearing by strain corresponding to the first 30 bins of sessions. The dose of protoveratrine B in mg/kg is shown on the y-axis.

FIG. 4 illustrates the chemical structures of protoveratrine A, protoveratrine B, veratramine, cevadine/veratrine, and veratrosine.

### DETAILED DESCRIPTION OF ILLUSTRATIVE EMBODIMENTS

Psychiatric and behavioral disorders according to the present invention include but are not limited to attention deficit/hyperactivity disorder (ADHD), attention deficit disorder (ADD), obsessive compulsive disorder, autism, pervasive developmental disorder, autism spectrum disorder, seizure disorder, anxiety, generalized anxiety, generalized anxiety disorder, panic attack, drug dependency withdrawal, sleep disorders, insomnia, and depression (including bipolar depression). In certain embodiments, the disorder is ADHD and/or ADD. In other embodiments, the disorder is depression, particularly bipolar depression, while in further embodiments the disorder is autism, pervasive developmental disorder, or autism spectrum disorder. In certain preferred embodiments, the disorder is anxiety, generalized anxiety or generalized anxiety disorder.

The diagnosis of psychiatric or behavioral disorders and evaluation of the various associated symptoms is well known to those skilled in the art and may be carried out by any of the known methods. Representative techniques can be found, for example, in the scientific literature, such as in Diagnostic and Statistical Manual of Mental Disorders, Fourth Edition. One of the diagnostic criteria for ADHD and ADD, for example, is the presence of six or more of the following symptoms of inattention for a period of six months such that it is maladaptive and inconsistent with developmental level: (i) failure to give close attention to details or making careless mistakes in school work, work, or other activities; (ii) difficulty sustaining attention in tasks or play activities; (iii) does not seem to listen when spoken to directly; (iv) does not follow through on instructions and fails to finish school work, chores, or duties in the workplace; (v) difficulty organizing tasks and activities; (vi) avoids, dislikes, or is reluctant to engage in tasks that require sustained mental effort; (vii) loses things necessary for tasks or activities; (viii) easily distracted by extraneous stimuli; and (ix) forgetful in daily activities. Another diagnostic criteria for ADHD is the evaluation of following symptoms of hyperactivity-impulsivity for a period of six months such that it is maladaptive and inconsistent with developmental level: (i) fidgets with hands or feet or squirms in seat; (ii) leaves seat in classroom or in other situation in which remaining in seat is expected; (iii) runs about or climbs excessively in situations in which it is inappropriate; (iv) difficulty playing or engaging in leisure activities quietly; (v) often “on the go” or acts as if “driven by a motor;” (vi) talks excessively; (vii) blurts out answers before questions have been completed; (viii) has difficulty awaiting turn; and (ix) interrupts or intrudes on others.

Generalized anxiety disorder, in turn, can be characterized by excessive worry, i.e., excessive concerns about real life concerns. Obsessive-compulsive disorder is characterized by recurrent obsessions or compulsions that are severe enough to be time consuming or cause marked distress or significant impairment. Obsessions are persistent ideas, thoughts, impulses or images that are experienced as intrusive and inappropriate and that cause marked anxiety or stress. Compulsions are repetitive behaviors or mental acts the goal of which is to prevent or reduce anxiety or distress, not to provide pleasure or gratification.

Autism spectrum disorder includes three separate diagnoses which include Asperger's syndrome, Pervasive Developmental Disorder (PDD) and autism. PDD is characterized by developmental delays of sociability, communication and use of imagination. Asperger's syndrome lacks the language and intelligence deficits normally associated with autism. Autism is characterized by severe communication impairments, social interaction deficits, and repetitive/stereotypic behaviors.

Addiction has been defined by the National Research Council as a state of periodic or chronic intoxication detrimental to the individual and produced by the repeated administration of a drug. The addicted individual develops a continuing craving for the drug and experiences withdrawal symptoms such as sweating, shivering, and anorexia if an attempt is made to discontinue drug use. The drug may be a “recreational” drug, for example heroin or cocaine, or it may be a therapeutic drug that is initially taken to relieve medical symptoms such as pain or insomnia.

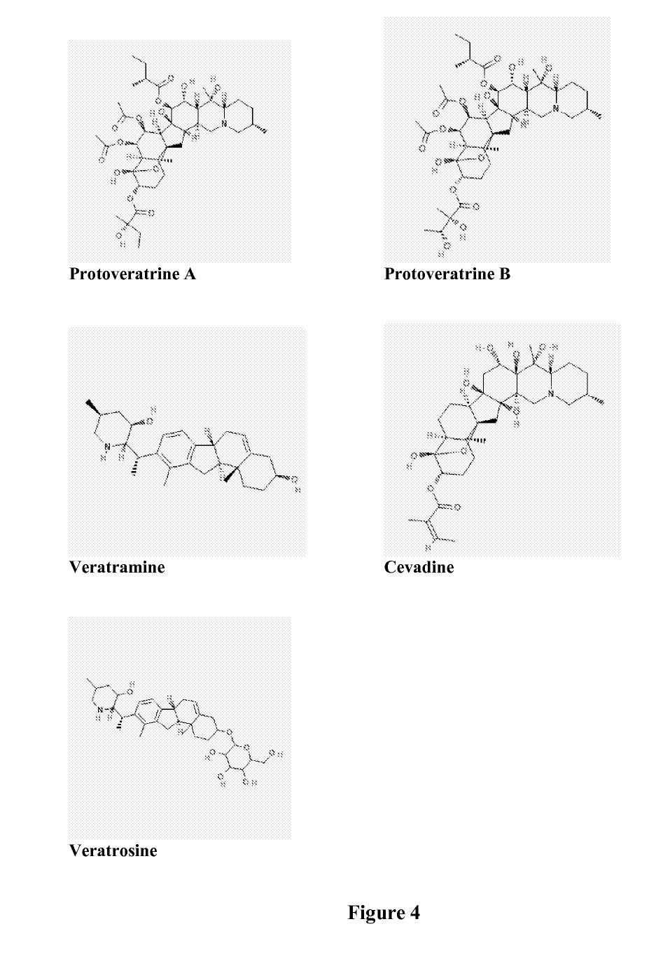

In accordance with the present invention, such disorders are treated through administration of an active agent that comprises protoveratrine A, protoveratrine B, cevadine, also known as veratrine, veratramine, veratrosine, or a combination thereof in an amount effective to lessen the severity or occurrence of at least one symptom indicative of the disorder. As used herein, each of these terms refers to the compound having the structure shown in FIG. 4. The dosage form can be administered through any of the modes known in the art, including but not limited to oral, transdermal, transmucosal, anal or pulmonary administration or by injection, e.g. subcutaneous, intravenous, intramuscular, intrathecal or intraperitoneal injection.

In certain embodiments, protoveratrine A is administered intravenously at a dosage that is between 0.0002 and 0.0025 milligrams per kilogram patient body weight (mg/kg). In preferred embodiments, the dosage is between 0.0002 and 0.0015 mg/kg. In yet further embodiments, the dosage is between 0.0003 and 0.0006 mg/kg. Preferably, this dosage is administered once daily.

In certain embodiments, protoveratrine B is administered intravenously at a dosage that is between 0.0002 and 0.0025 milligrams per kilogram patient body weight (mg/kg). In preferred embodiments, the dosage is between 0.0002 and 0.0014 mg/kg. In yet further embodiments, the dosage is between 0.0004 and 0.0008 mg/kg. Preferably, this dosage is administered once daily.

In certain embodiments, cevadine is administered intravenously at a dosage that is between 0.001 and 0.03 milligrams per kilogram patient body weight (mg/kg). In preferred embodiments, the dosage is between 0.001 and 0.018 mg/kg. In yet further embodiments, the dosage is between 0.002 and 0.004 mg/kg. Preferably, this dosage is administered once daily.

In certain embodiments, veratramine is administered intravenously at a dosage that is between 0.01 and 0.18 milligrams per kilogram patient body weight (mg/kg). In preferred embodiments, the dosage is between 0.01 and 0.09 mg/kg. In yet further embodiments, the dosage is between 0.02 and 0.04 mg/kg. Preferably, this dosage is administered once daily.

In certain embodiments, veratrosine is administered intravenously at a dosage that is between 0.01 and 0.18 milligrams per kilogram patient body weight (mg/kg). In preferred embodiments, the dosage is between 0.01 and 0.09 mg/kg. In yet further embodiments, the dosage is between 0.02 and 0.04 mg/kg. Preferably, this dosage is administered once daily.

In certain embodiments, protoveratrine A is administered intraperitoneally or intramuscularly at a dosage that is between 0.001 and 0.008 milligrams per kilogram patient body weight (mg/kg). In preferred embodiments, the dosage is between 0.0012 and 0.0045 mg/kg. In yet further embodiments, the dosage is between 0.0014 and 0.0022 mg/kg. Preferably, this dosage is administered once daily.

In certain embodiments, protoveratrine B is administered intraperitoneally or intramuscularly at a dosage that is between 0.001 and 0.006 milligrams per kilogram patient body weight (mg/kg). In preferred embodiments, the dosage is between 0.0014 and 0.004 mg/kg. In yet further embodiments, the dosage is between 0.0018 and 0.0023 mg/kg. Preferably, this dosage is administered once daily.

In certain embodiments, cevadine is administered intraperitoneally or intramuscularly at a dosage that is between 0.004 and 0.1 milligrams per kilogram patient body weight (mg/kg). In preferred embodiments, the dosage is between 0.005 and 0.04 mg/kg. In yet further embodiments, the dosage is between 0.007 and 0.012 mg/kg. Preferably, this dosage is administered once daily.

In certain embodiments, veratramine is administered intraperitoneally or intramuscularly at a dosage that is between 0.04 and 0.45 milligrams per kilogram patient body weight (mg/kg). In preferred embodiments, the dosage is between 0.05 and 0.28 mg/kg. In yet further embodiments, the dosage is between 0.07 and 0.12 mg/kg. Preferably, this dosage is administered once daily.

In certain embodiments, veratrosine is administered intraperitoneally or intramuscularly at a dosage that is between 0.04 and 0.45 milligrams per kilogram patient body weight (mg/kg). In preferred embodiments, the dosage is between 0.05 and 0.28 mg/kg. In yet further embodiments, the dosage is between 0.07 and 0.12 mg/kg. Preferably, this dosage is administered once daily.

In certain embodiments, protoveratrine A is administered orally at a dosage that is between 0.1 and 0.9 milligrams per kilogram patient body weight (mg/kg). In preferred embodiments, the dosage is between 0.12 and 0.4 mg/kg. In yet further embodiments, the dosage is between 0.15 and 0.3 mg/kg. Preferably, this dosage is administered once daily.

In certain embodiments, protoveratrine B is administered orally at a dosage that is between 0.1 and 1.0 milligrams per kilogram patient body weight (mg/kg). In preferred embodiments, the dosage is between 0.12 and 0.6 mg/kg. In yet further embodiments, the dosage is between 0.15 and 0.4 mg/kg. Preferably, this dosage is administered once daily.

In certain embodiments, cevadine is administered orally at a dosage that is between 0.2 and 1.5 milligrams per kilogram patient body weight (mg/kg). In preferred embodiments, the dosage is between 0.2 and 0.9 mg/kg. In yet further embodiment, the dosage is between 0.3 and 0.5 mg/kg. Preferably, this dosage is administered once daily.

In certain embodiments, veratramine is administered orally at a dosage that is between 0.4 and 3.0 milligrams per kilogram patient body weight (mg/kg). In preferred embodiments, the dosage is between 0.6 and 1.8 mg/kg. In yet further embodiments, the dosage is between 0.8 and 1.2 mg/kg. Preferably, this dosage is administered once daily.

In certain embodiments, veratrosine is administered orally at a dosage that is between 0.4 and 3.0 milligrams per kilogram patient body weight (mg/kg). In preferred embodiments, the dosage is between 0.6 and 1.8 mg/kg. In yet further embodiments, the dosage is between 0.8 and 1.2 mg/kg. Preferably, this dosage is administered once daily.

The present invention also provides a pharmaceutical dosage form comprising protoveratrine A in an amount that is no more than about 0.9 mg/kg. The present invention also provides a pharmaceutical dosage form comprising protoveratrine B in an amount that is no more than about 1.0 mg/kg. Preferably, this dosage is administered once daily.

In some cases, a physician may determine that it is beneficial to treat the patient by co-administering other drugs that also find use in treating psychiatric and behavioral disorders. Drugs commonly used to treat ADHD include methylphenidate, amphetamines, and Atomoxetine HCl. Drugs commonly used to treat anxiety comprise fluoxetine, benzodiazepines, such as Xanax, Klonopin, Valium, ativan, antidepressants, or beta-blockers. Drugs commonly used to treat obsessive compulsive disorder include Clomipramine, fluvoxamine, fluoxetine, Sertraline, Paroxetine, Citalopram. Drugs commonly used to treat autism, pervasive developmental disorder and autism spectrum disorder comprise antidepressants, antipsychotics, and all drugs used for ADHD.

Active agents according to the invention can be administered in virtually any dosage form known in the art. Although the active agents can be present in the dosage form in pure or substantially pure form, they are more typically present in the form of a pharmaceutical composition with one or more physiological acceptable carriers, diluents, excipients, or auxiliaries depending largely upon the route of administration chosen. Pharmaceutical compositions comprising the active agents of the invention may be manufactured by means of conventional mixing, dissolving, granulating, dragee-making, levigating, emulsifying, encapsulating, entrapping or lyophilizing processes.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hank's solution, Ringer's solution, or physiological saline buffer. The solution may contain formulatory agents such as suspending, stabilizing and/or dispersing agents, such as 0.9% sodium chloride.

Alternatively, the compounds may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the compounds can be readily formulated by combining the active agent with pharmaceutically acceptable carriers well known in the art. Such carriers enable the agent to be formulated as tablets, pills, dragees, capsules, liquid gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium, carboxymethylcellulose, and/or polyvinylpyrrolidone (PVP); granulating agents; and binding agents. If desired, disintegrating agents may be added, such as the cross-linked polyvinylpyrrolidine, agar, or alginic acid or a salt thereof such as sodium alginate.

If desired, solid dosage forms may be sugar-coated or enteric-coated using standard techniques.

For oral preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the active agents may take the form of tablets, lozenges, etc. formulated in conventional manner.

For administration by inhalation, the active agents of the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and suitable powder base such as lactose or starch.

The active agents may also be formulated in rectal or vaginal compositions such as suppositories or retention enemas, e.g., containing conventional suppository bases such as cocoa butter or other glycerides.

In addition to the formulations described previously, the active agents may also be formulated as a depot preparation. Such long acting formulations may be administered by implantation (for example subcutaneously or intramuscularly) or by intramuscular injection. Thus, for example, the agents may be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery system may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Because certain of the active agents of the invention may contain charged side chains or termini, they may be included in any of the above-described formulations as the free acids or bases or as pharmaceutically acceptable salts. Pharmaceutically acceptable salts are those salts which substantially retain the antimicrobial activity of the free bases and which are prepared by reaction with inorganic acids. Pharmaceutical salts tend to be more soluble in aqueous and other protic solvents than are the corresponding free base forms.

The active agents of the invention will preferably be used in an amount effective to lessen the severity or occurrence of at least one symptom indicative of a psychiatric or disorder. Determination of an effective amount is well within the capabilities of those skilled in the art, especially in light of the detailed disclosure provided herein. Initial dosages can be estimated from in vivo data. e.g. animal models, using techniques that are well known in the art. Dosage amount and interval may be adjusted individually to provide plasma levels of the active agents which are sufficient to maintain the desired therapeutic effect. Therapeutically effective serum levels may be achieved by administering multiple doses each day.

In cases of local administration or selective uptake, the effective local concentration of the active agents might not be related to plasma concentration. One having skill in the art will be able to optimize therapeutically effective local dosages without undue experimentation.

The amount of compound administered will, of course, be dependent on the patient being treated, on the patient's weight, the severity of the affliction, the manner of administration and the judgment of the prescribing physician.

The following examples are provided to describe the embodiments described herein with greater detail. They are intended to illustrate, not to limit, the embodiments.

### Example 1

The effects of Protoveratrine B (PVB) on locomotor activity were determined using an open-field apparatus (High Density Cage Rack System; Kent Scientific Corporation). Adult spontaneously hypertensive rats (SHR) and adult Wistar Kyoto rats (WKY) were purchased from Charles River Laboratories. Following three baseline sessions (previous research has indicated that strain differences in activity are revealed after several sessions); rats were injected (i.p.) with drug 5-min prior to being placed in the open-field apparatus. Sessions lasted 30-minutes and were separated by at least one day to diminish the likelihood of habituation to the apparatus. Three doses of PVB were investigated based upon the blood-pressure phase of the study: 0.01, 0.02 & 0.04 mg/kg.

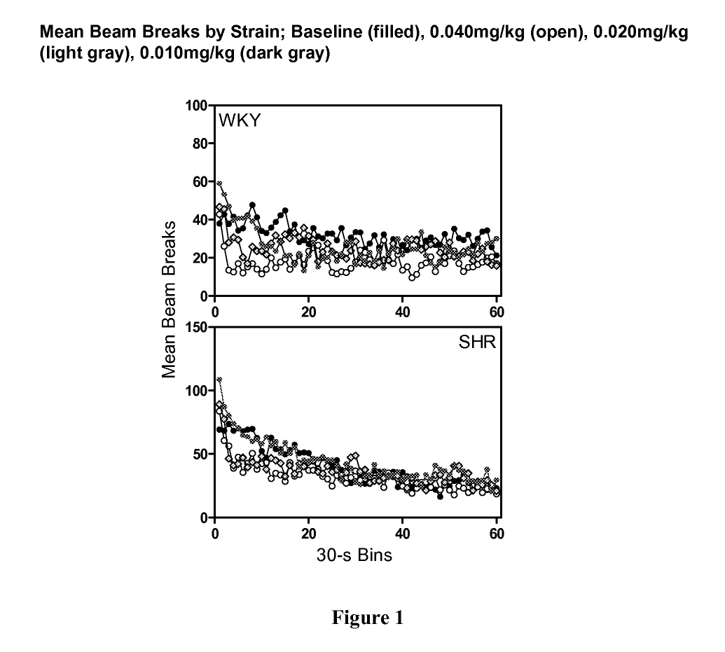

FIG. 1 shows the average activity level (as defined by the number of beam breaks) as a function of time. The number of beam breaks was averaged across subjects and into 30-s bins. The upper panel represents the WKY control rats (N=8); the bottom panel represents the SHR rats (N=8). The different plots represent the baseline and different doses of PVB. Several details can be gleaned from the data. First, the SHRs are much more active than the WKYs at the beginning of the session (note the differences in the range of the ordinate). Second, activity levels in the SHRs appear to drop off precipitously throughout the session and eventually converge with the activity levels seen in the WKYs. Third, PVB appears to have decreased activity levels in both strains at least initially in the first half of the session. Fourth, the largest dose (0.04 mg/kg) of PVB appears to have had a greater decrease in activity than the lowest dose (0.01 mg/kg). Fifth, the 0.01 mg/kg dose appears to have reduced activity from baseline in WKYs, but did not affect activity in SHRs.

### Example 2

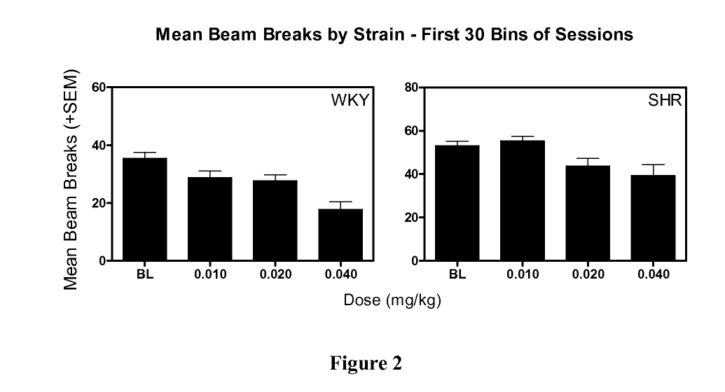

To better comprehend the effects of dose on locomotor activity, a dose-response graph was constructed for each rat strain. To construct these graphs, the number of beam breaks that occurred in the first thirty bins (first 15 min) of all rats within a strain was averaged and plotted as a function of dose. The resulting graphs, shown in FIG. 2, represent the effects of PVB on activity within the first 15 minutes post injection.

As can be seen, SHRs appear to be more active than WKYs. PVB reduced the mean number of beam breaks for both strains although this shape of the dose-response functions differed between the strains. The WKYs were affected by the lowest dose; whereas this dose appears to have had no activity-decreasing effect in the SHRs. Further, the largest dose appears to have caused a greater reduction in activity levels in the WKYs. Taken together, this finding suggests that PVB might be slightly more potent in WKY than in SHR rats.

### Example 3

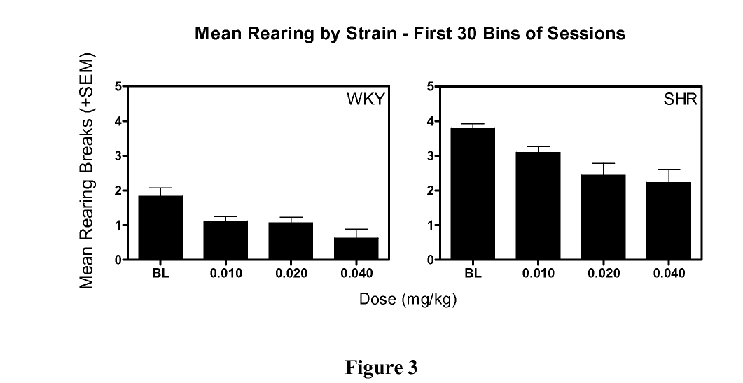

Another measure of activity level is rearing (measured by beam breaks of an infrared plane approximately 5″ above the floor of the chamber). Rearing data were analyzed in the same way as the horizontal beam breaks. The results are illustrated in FIG. 3. SHRs appear to have been more active than WKYs. Moreover PVB appear to have reduced rearing in both strains. Unlike the horizontal activity, rearing activity in SHRs was decreased by the 0.01 mg/kg dose.

What is claimed:

1. A method comprising the steps of:
identifying a patient suffering from a psychiatric disorder or a behavioral disorder; and
administering to said patient an active agent that comprises protoveratrine A, protoveratrine B, cevadine, veratramine, veratrosine, or a combination thereof in an amount effective to lessen the severity or occurrence of at least one symptom indicative of said disorder.

2. The method of claim 1 wherein the disorder is ADHD, ADD, obsessive compulsive disorder, autism, pervasive developmental disorder, autism spectrum disorder, seizure disorder, anxiety, panic attack, drug addiction withdrawal, sleep disorders, insomnia, depression or bipolar depression.

3. The method of claim 1 wherein the disorder is ADHD.

4. The method of claim 1 wherein the disorder is ADD.

5. The method of claim 1 wherein the disorder is depression or bipolar depression.

6. The method of claim 1 wherein the disorder is anxiety, generalized anxiety or generalized anxiety disorder.

7. The method of claim 1 wherein the disorder is drug dependency withdrawal.

8. The method of claim 1 wherein the disorder is autism, pervasive developmental disorder, or autism spectrum disorder.

9. The method of claim 1 wherein the disorder is a sleep disorder or insomnia.

10. The method of claim 1 wherein the disorder is resistance to sedation.

11. The method of claim 1 wherein the active agent comprises protoveratrine A.

12. The method of claim 1 wherein the active agent comprises protoveratrine B.

13. The method of claim 1 wherein the protoveratrine A is administered intravenously at a dosage that is between 0.0002 and 0.0025 mg/kg.

14. The method of claim 1 wherein the protoveratrine B is administered intravenously at a dosage that is between 0.0002 and 0.0025 mg/kg.

15. The method of claim 1 wherein the protoveratrine A is administered intraperitoneally or intramuscularly at a dosage that is between 0.001 and 0.008 mg/kg.

16. The method of claim 1 wherein the protoveratrine B is administered intraperitoneally or intramuscularly at a dosage that is between 0.001 and 0.006 mg/kg.

17. The method of claim 1 wherein the protoveratrine A is administered orally at a dosage that is between 0.1 and 0.9 mg/kg.

18. The method of claim 1 wherein the protoveratrine B is administered orally at a dosage that is between 0.1 and 1.0 mg/kg.

19. The method of claim 1 further comprising examining said patient after said administration and determining any extent to which said protoveratrine A, protoveratrine B, cevadine, veratramine, veratrosine, or combination thereof affected at least one symptom indicative of said disorder.

20. A pharmaceutical dosage form comprising protoveratrine A in an amount that is no more than 0.9 mg/kg.

21. A pharmaceutical dosage form comprising protoveratrine B in an amount that is no more than 1.0 mg/kg.
